(19) United States
(12) Patent Application Publication
(10) Pub. No.: US 2021/0008293 A1
(43) Pub. Date: Jan. 14, 2021

(54) DEVICE FOR PREPARING A DOSE OF MEDICAMENT ON DEMAND

(71) Applicant: Unither Pharmaceuticals, Amiens (FR)

(72) Inventor: Marc Maury, Saint Medard en Jalles (FR)

(21) Appl. No.: 16/979,399

(22) PCT Filed: Mar. 11, 2019
(86) PCT No.: PCT/EP2019/055999

§ 371 Date: Sep. 9, 2020

(30) Foreign Application Priority Data
Mar. 9, 2018 (FR) 1870257

(51) Int. Cl. A61M 5/315; A61M 5/24

(52) U.S. Cl. CPC A61M 5/31548; A61M 5/24; A61M 2005/3126; A61M 2202/04; A61M 2202/064

(57) ABSTRACT

The invention concerns a device for preparing a dose of medicament on demand, comprising at least one receptacle (10), at least one cartridge (12) of a medicinal composition intended to enter said at least one receptacle, the medicinal composition being in liquid or solid powder form, as well as dosing means (14) for dosing said medicinal composition, monitoring means (16) for monitoring the dose of the medicinal composition delivered and control means (18) for controlling the at least one cartridge (12), the dosing means (14) and the monitoring means (16).

The present invention relates to a device for preparing a dose of medicament on demand. The administration of medicaments from containers having a volume corresponding to a number of doses poses a number of problems.

Underdosing medicaments leads to ineffective action on the patient and overdosing may have serious consequences.

Moreover, administration is within ranges, when said ranges exist, in particular for children. For adults or elderly people, the existing pharmaceutical forms such as tablets or capsules do not permit adaptation to the physiology of each patient.

With children, the dosage is necessarily refined according to weight.

In pediatric ranges, it is customary to administer one or more variable doses from a multi-dose container, using a dose administration utensil such as a spoon or a dropper.

There is therefore a risk of error in the number of doses or in the volume if the dosing utensil used is over- or under-full, particularly as regards the formation of a meniscus due to the viscosity of the concentrated product.

Moreover, if the dosing utensil is lost or interchanged among a number of medicaments, it is not unusual to find that parents or elderly people use another utensil of a different volume.

Every year this leads to reports of accidents concerning medicaments, even those normally known for their safety in use.

Safe administration should therefore prioritized in any innovative device.

Furthermore, the medicament contained in a multi-dose bottle may be used, but the medicinal content is in contact with the air, which may cause oxidation of air-sensitive compounds on the one hand and/or contamination by microorganisms on the other. The medicament is also in contact with the dosing utensil which receives said medicament, which, as is known, is not always thoroughly cleaned.

Medicaments are also available in powder form for dissolving but in this case said medicaments are in sachets with limited adjustment possibilities apart from increasing the number of sachets containing very small quantities and if said medicaments are in multi-dose bottles, the problem remains of overdosing or underdosing for powder that must be dissolved.

Moreover, the problem arises of the quality of the dilution of the powder or liquid in water, for example. Good-quality water is required, not necessarily water from the drinking water network, which although drinkable, is not always suitable for infants in particular. The natural or supplemented dilution liquid is considered a medicinal dilution composition and could also be of interest.

Finally, some medicaments have a very limited shelf life once opened, which leads at best to incomplete use of the contents of the multi-dose container but also to the use of medicaments that have been chemically or microbiologically altered and are therefore not suitable for consumption.

The present invention relates to a safety device with a plurality of inputs and verifications so as to allow appropriate, simple dosage with no risk of error for the person administering the medicament. Moreover, the device prevents all contact of the contents of the multi-dose container with the air. Said device may also provide for the reconstitution of a medicament in liquid form from a medicinal composition in powder form or for the dilution of a liquid medicinal composition. The device prevents any contamination because all the elements are for dedicated, contactless use, and overcomes the need for any cleaning of any portion of the device.

This type of device may also be very attractive in a professional setting such as in establishments that receive elderly people, hospitals or clinics.

A device for preparing food supplements, i.e. non-medicinal products, is known from patent application US2016/0280454 which uses a base with water injected into a receptacle provided with a mixer. Said device perfectly illustrates the prior art in proposing a device in which a cartridge with chambers is provided, each chamber containing a dose of powdered product for dispensing. The carousel is therefore specific to the product to be dispensed but always dispenses the required dose, the only option being to increase by a multiple the number of doses delivered. However, no precise adjustment is possible. Furthermore, the mixer cup receiving the powder and the dilution water comprises a mixer which is very specific and must be re-used, thus leading inevitably to imperfect cleaning, particularly where the paddles are integral. The chambers used necessarily contain powder residue and remain open during rotation. However, the programming may last a number of weeks even though the powder residue is subject to hygroscopy from the air, which would not be compatible with the preparation and dispensing of medicaments. In particular it is noted that a change of product is linked to a change of carousel but the mixer cup is retained. It is also very clear that the dispenser in this patent application is associated with a very specific carousel, which is necessarily very expensive and not compatible with the desire to reduce packaging by using very simple containers that are not material intensive and have large volumes from which a suitable dosage of medicaments are to be dispensed, including for infants.

Also known is document US2013/0068790 which describes an arrangement for a medicament dispenser which has the same aims as the present invention but uses totally different means. The device is of unusual complexity as it is intended for professionals and aims to dispense a particular product, morphine. The tank is not only flexible so as to be sensitive to pressure but also comprises an airbag device which ensures the product is neutralized if accidental opening or an external event occurs so as to prevent free access to the product. Said device has a high level of security and is not intended for domestic applications. Said device is the antithesis of a simple, reliable, inexpensive dispenser accessible to all.

The device according to the present invention, which overcomes the drawbacks of the dispensers of the prior art in particular and provides a solution to the problems referred to above is now described with reference to the accompanying drawings, in which the different figures show a non-limiting, diagrammatic embodiment.

Said different figures show:

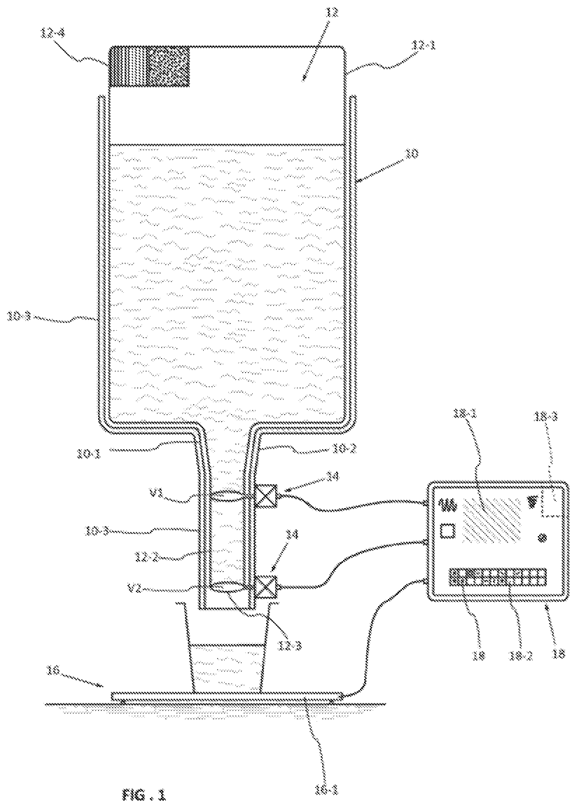

FIG. 1: a view in cross section of the device,

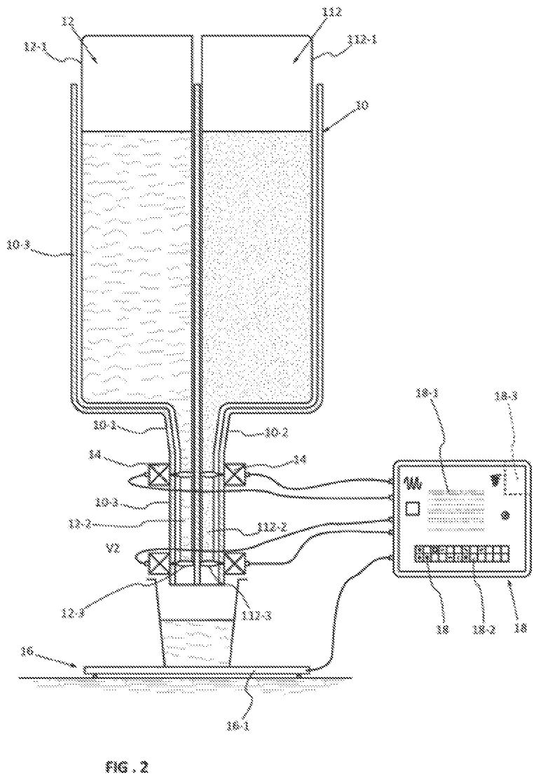

FIG. 2: a view of a variant with a combined arrangement incorporating a second container for a dilution liquid composition.

FIG. 1 shows the device according to the present invention which comprises at least one receptacle 10 for a cartridge 12 of a medicinal composition in liquid or solid powder form, as well as dosing means 14, monitoring means 16 associated with the dose of the prescribed medicinal composition, and control means 18 for the entire device.

The receptacle 10 comprises reception and removal means 10-1 for the cartridge 12, in this case a funnel 10-2 with vertical walls 10-3 and a truncated bottom, provided at the lower end with a guide tube 10-5 receiving the dosing means 14.

The cartridge 12 comprises a main volume 12-1 made of a flexible or rigid material, in which a given volume of a medicinal composition in powder or liquid form is placed. The term “powder” refers to powder and also granules.

Said given volume corresponds to a number of doses.

Said main volume is extended by a flexible conduit 12-2 provided with closing/opening means 12-3, in this case and in this specific embodiment, said closing/opening means 12-3 are made up of two valves, V1 and V2. Said conduit comprises delivery means at the end such as an end valve. Said flexible conduit 12-2 is rigidly connected to the cartridge and intended for single use with the pouch.

Said flexible conduit is intended to cooperate with the guide tube 10-5 of the receptacle.

The upper valve V1 is close to the actual container and the valve V2 is positioned at the free end of the flexible conduit.

The term “valve” refers to any means of stopping/releasing the flow of the fluid or solid powdered medicinal composition through said flexible conduit, allowing sealed closure. Said valves may be diaphragm valves, ball valves or groove-and-blade duckbill valves.

Each cartridge 12 has an identifier 12-4 with fixed or writable data, dosage instructions associated with use-by dates, opening and hence initial operation detectors, etc. Said identifier may advantageously take the form of an RFID chip or a high-density encoding optical bar code such as a DataMatrix code.

Said identifier is connected to the control means 18, which are suitable for reading said identifier and controlling the device according to the present invention using the data read.

The dosing means 14 consist of means intended to operate the closing/opening means 12-3 of the cartridge 12.

Thus, if the closing/opening means 12-3 of the cartridge 12 comprise diaphragms, the dosing means 14 comprise means for applying pressure and opening the diaphragms separately or simultaneously, for example by peripheral external mechanical pressure like a peristaltic pump with a roller on the flexible conduit so as to squeeze said conduit in order to propel the contents of the pipe inside said pipe. The rolling duration, rolling speed and diameter of the conduit allow the required dosage to be delivered. It will be noted that said volumes are independent of external conditions and very simple, and the medicament is not in contact with the outside environment.

If the closing/opening means 12-3 of the cartridge 12 are balls, the dosage means 14 may comprise movable, magnetized means that can be moved in translation, allowing the balls to move and cause a fluid or solid powder flow adjusting the required dosage.

If the closing/opening means 12-3 of the cartridge 12 comprise duckbill groove-and-blade valves, the dosage means 14 comprise means for applying pressure that allow said groove with blades to be deformed and thus cause a fluid or solid powder flow adjusting the required dosage.

The cartridge may also be a flexible pouch provided with a distributor valve. The medicinal composition is contained in the flexible pouch. A flexible sampling tube is immersed in the pouch beneath the valve. The distributor valve ensures that a given volume of medicinal composition is delivered, the volume dispensed being proportional to the number of times pressure is applied to the distribution valve. The flexible conduit 12-2 is in this case rigidly connected to the cartridge at the outlet of the distributor valve and cannot be separated. The number of times mechanical pressure is applied to the distributor valve corresponds to the dose of medicinal composition to be dispensed. The flexible conduit 12-2 may also, preferably, comprise an end valve forming the final delivery means at the outlet of the flexible conduit 12-2 to protect from the air and thus preserve the product remaining in the flexible conduit between two successive dispensing events, as above.

If the closing/opening means 12-3 of the cartridge 12 comprise a distribution valve, the dosing means 14 comprise a motor with an eccentric or an electromagnetic piston for example, so as to apply mechanical pressure for the required number of times.

Said dosing means 14, in whatever embodiment, are connected to the control means 18.

The means 16 for monitoring the amount delivered by the means 14 for dosing the dispensed medicinal composition comprise in a first embodiment weighing means 16-1 positioned beneath the container with an automatic tare function for any type of ingestion vessel, such as a glass. Thus, the measured weight deducted from the tare weight corresponds to the weight of liquid or powder dispensed into a vessel. The dilution liquid, if present, may also be monitored after dosing in the same way.

The monitoring means 16 may comprise optical means 16-2 in the form of a contactless volume detection function. Such optical means can detect the amount of a tracer included in the medicinal composition at a given concentration in order to detect the tracer as it passes.

The monitoring means 16 are connected to the control means 18.

Said control means 18 comprise display means 18-1, such as a screen, input means 18-2, such as a touch keypad, means for storing and implementing software 18-3, such as a processor.

The software storage and implementation means 18-3 are suitable for receiving the signals, commands, information from or to the cartridge 12, the dosing means 14 and/or the monitoring means 16.

Thus, a way of operating the device by an operator such as a parent, a member of a medical staff or a patient may be described.

If the operator wishes to deliver a dose of medicinal composition, said operator introduces a container with a single volume corresponding to a number of doses into the receptacle. Normally, if this is the first use, a safety closure must previously have been removed.

The operator inserts the flexible conduit 12-2 into the guide tube 10-5 if necessary. The flexible conduit 12-2 is either provided with integral opening/closing means, or inserted between the rollers of a peristaltic pump, or immobilized in a guide so that an optical detector can operate and control the magnetized valves, or alternatively the flexible conduit is simply positioned after the outlet of the distributor valve.

From the identifier 12-4, the control means 18 determine the type of medicinal composition and all the associated parameters such as date of opening, potential use-by date and administration time compared to delivery time, and record all the elements so as to allow event traceability.

The operator uses the screen to input data relating to the patient: age, sex, weight and more generally all parameters required for correct administration. These questions are posed by the control means 18 and originate more particularly from the software.

Then the device delivers a dose when triggered by the operator, provided in particular that the weighing means have detected a vessel, the tare being calculated immediately.

The medicinal composition is delivered through the flexible conduit by the operation of the two valves V1 and V2 and/or by the volumetric powder dosing system, by operating the distributor valve or alternatively by rotating the rollers of the peristaltic pump, depending on the type of dosage means 14 adopted.

The dose of medicinal composition delivered may be monitored by optical means, if present and operational, depending on the medicinal composition and/or by the weighing means.

The dose of medicinal composition is therefore perfectly tailored to the patient who is to take it.

It is however possible to produce a time-adjusted dose to compensate for forgetting to take a dose or for taking said dose early or late.

The device according to the present invention also provides various security means such as preventing delivery of the medicinal composition at a shorter interval between two doses than the recommended one, this being managed by the control means 18. Similarly, in the event of an electricity supply failure, the control means provide for storing the time in order to reset the schedules and take account of the length of the power cut. As this is a medicinal composition, the control means record and store all the history in order to determine cartridge changes, outages, etc.

If the medicinal composition is in solid or liquid form but highly viscous or highly concentrated, the solid or liquid dose is dispensed but must be diluted, which the control means 18 prompt the operator to do via the screen.

Preferably, the device may provide for a second cartridge 112 with its own identifiers, identical in design to the first cartridge 12 and containing a dilution liquid which may be water or a composition of suitable quality or alternatively a second medicinal composition and means of dosing the liquid in said second cartridge.

Identical elements of said variant in FIG. 2 are retained. The elements of the second cartridge are identical but the reference numerals are increased by 100.

Said second cartridge and the associated receptacle, and the dosing means are also connected to the control means 18.

In this case, the second cartridge 112 delivers the dilution liquid either for a highly viscous liquid composition or for a powder, so as to produce a drinkable composition having a determined volume, dilution and/or viscosity and suitably convenient to use.

Said two compartments allow compositions to be dispensed that may prove incompatible or unstable in conjunction with one another during prolonged storage. In the case of the present invention, the composition is produced at the last moment so that conservation or instability problems cannot develop.

The two compartments may be powder/liquid or liquid/liquid.

Similarly, in addition to or instead of weighing, the optical detection means may optically quantify the volume of medicament delivered, for example by means of a fluorescent tracer or a sorbent of a particular wavelength. Advantageously, the constituent elements of the device are assembled in an ergonomic casing, not shown, of suitable aesthetic appearance.

However, this is not part of the present invention as assembly in a casing is within the scope of a person skilled in the art, and aesthetics are extraneous to the technique described.

1. Device for preparing a dose of medicament on demand, comprising at least one receptacle (10), at least one cartridge (12, 112) of a medicinal composition or dilution composition intended to enter said at least one receptacle, the medicinal composition being in liquid or solid powder form, as well as dosing means (14) for dosing said medicinal composition, monitoring means (16) for monitoring the dose of the medicinal composition delivered and control means (18) for controlling the at least one cartridge (12, 112), the dosing means (14) and the monitoring means (16).

2. Device for preparing a dose of medicament on demand according to claim 1, characterized in that each at least one cartridge (12, 112) comprises an identifier (12-4) with fixed or writable data, chosen from the dosing instructions associated with the medicinal composition with use-by dates, opening and initial operation detectors.

3. Device for preparing a dose of medicament on demand according to claim 2, characterized in that the identifier (12-4) is an RFID chip.

4. Device for preparing a dose of medicament on demand according to claim 2, characterized in that the identifier (12-4) is a high-density encoding optical bar code.

5. Device for preparing a dose of medicament on demand according to claim 1, characterized in that the cartridge (12, 112) comprises a main volume (12-1) extended by a flexible conduit (12-2) provided with delivery means at the end thereof.

6. Device for preparing a dose of medicament on demand according to claim 5, characterized in that each cartridge (12, 112) comprises closing/opening means (12-3).

7. Device for preparing a dose of medicament on demand according to claim 4, characterized in that the receptacle (10) comprises reception and removal means (10-1) for the cartridge (12), provided at the lower end with a guide tube (10-5) intended to receive the flexible conduit (12-2) of the cartridge (12).

8. Device for preparing a dose of medicament on demand according to claim 6, characterized in that the dosing means (14) comprise means for operating the closing/opening means (12-3) of the cartridge (12).

9. Device for preparing a dose of medicament on demand according to claim 1, characterized in that the means (16) for monitoring the dosage of the medicinal composition dispensed by the dosing means comprise weighing means (16-1).

10. Device for preparing a dose of medicament on demand according to claim 1, characterized in that the monitoring means (16) comprise contactless optical volume detection means (16-2).

11. Device for preparing a dose of medicament on demand according to claim 1, characterized in that the control means (18) comprise display means (18-1), input means (18-2), software storage and implementation means (18-3).
